(19) United States
(12) Patent Application Publication
(10) Pub. No.: US 2017/0247335 A1
(43) Pub. Date: Aug. 31, 2017

(54) FLUORINATED PYRIDAZIN-3-ONES FOR THE USE THEREOF IN THE TREATMENT OF LUNG DISEASES

(71) Applicants: UNIVERSITE DE REIMS CHAMPAGNE-ARDENNE, Reims (FR); INSERM (INSTITUT NATIONAL DE LA SANTE ET DE LA RECHERCHE MEDICALE), Paris (FR); UNIVERSITE DE ROUEN, Mont Saint-aignan (FR)

(72) Inventors: Stephane GERARD, Fismes (FR); Jean-Philippe BOUILLON, Fresquiennes (FR); Azzaq BELAAOUAJ, Muizon (FR); Eric HENON, Reims (FR)

(21) Appl. No.: 15/523,378

(22) PCT Filed: Oct. 30, 2015
(86) PCT No.: PCT/FR15/52929

§ 371 Date: Apr. 29, 2017

(30) Foreign Application Priority Data
Oct. 31, 2014 (FR) 1460501

(51) Int. Cl. C07D 237/14

(52) U.S. Cl. CPC C07D 237/14

(57) ABSTRACT

The present invention concerns compounds belonging to the family of fluorinated pyridazin-3-ones, for the use thereof in the treatment of broncho-pulmonary conditions. In compounds having a formula, or a pharmaceutically acceptable salt of the compound, the formula includes R1 representing H, an alkyl, an aryl or a heteroaryl; either E2 and E3 representing, separately from each other, H, an alkyl, an aryl or a heteroaryl, or R2 and R3 being bridged within a same cycle or via several cycles; and F representing CF₃, (CF₂)nCF₃ or CF₂H, with n representing an integer of between 1 and 7.

### CROSS-REFERENCE TO RELATED APPLICATIONS

See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

1. Field of the Invention

The present invention relates to the field of compounds belonging to the family of the fluorinated pyridazinones.

2. Description of Related Art Including Information Disclosed Under 37 CFR 1.97 and 37 CFR 1.98.

The present invention will find its application mainly in the field of treatment against chronic obstructive lung diseases (COLM), notably obstructive chronic broncho-pneumo pathologies (OCBP), asthma and cystic fibrosis.

These affections are in regular increase in developed countries. As regards France, more than 10% of patients are affected with OCBP, and this generates significant health expenses, estimated at about 3.5 billion euros per year. Asthma affects 3.5 million persons, of which a third are less than 15 years old. Cystic fibrosis would affect one newly born over 4,200. It is estimated that 200 children are born each year in France with cystic fibrosis.

In spite of spectacular progress of these last 25 years in the efficiency and the use of drugs to be taken as basic treatments, it is estimated that about seven persons decease per day from this type of pathologies. The non-observance, by the patient, of the prescriptions of his/her physician, is one of the reasons put forward for explaining the significant number of daily deaths related to these lung affections. Indeed, these are generally long term treatments which have to be taken often daily, and the follow up of which is obligatory, especially for children.

Traditionally, for healing these types of diseases, the treatments of a first intention are essentially a bronchodilator compounds likes Salbutamol (Ventolin®), Terbutaline (Bricanyl®), inhaled corticoids (Flixotide®) or other beta-agonist with a long duration of action, also known under the name of BALA, like Salbutamol (Servant®).

However, it is necessary to conduct research in order to disclose novel bronchodilator compounds which are more efficient and easier to use.

Further, these treatments also have the drawback of requiring a follow-up of the patient which may appear to be constraining, notably for children and pre-adolescents.

Thus, research was conducted in order to attempt to find a remedy to these problems and to propose alternatives to traditional treatments.

This research was able to show that the inhibitor compounds of phosphodiesterase of type IV (abbreviated as PDE4) are particularly promising for treating these types of pathologies.

More specifically, the compounds having a pyridazine-3-one unit, such as zardaverine and its analogues, proved to be interesting in the treatment of COLD.

However, such compounds may have some toxicity associated with a lack of selectivity and, consequently be at the origin of undesirable secondary effects, such as nausea or diarrhoeas, which should be minimised for the comfort of the patients.

Further, it is also interesting to propose compounds having a further improved efficiency as compared with the molecules which have already been proposed in the state of the art.

In patent document EP 1 373 259, a compound belonging to the family of pyridazine-3-ones is further known and having the following formula:

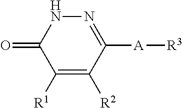

However, such a compound mandatorily includes a group A which represents a sulphur atom S, a sulphur oxide group SO or a sulphur dioxide group SO₂.

Further, this compound is intended for the treatment of ischemia of the heart tissue in mammals or further for the treatment of complications of diabetes in mammals, like for example diabetic neuropathy, diabetic nephropathy, etc.

The invention provides the possibility of overcoming the diverse drawback of the start of art by proposing a novel compound, belonging to the family of fluorinated pyridazine-3-ones, and allowing an efficient treatment of certain pathologies notably affecting the lungs, while minimizing in a particularly interesting way the aforementioned undesirable secondary effects traditionally encountered with this type of compound.

### BRIEF SUMMARY OF THE INVENTION

For this purpose, the present invention relates to a compound belonging to the family of fluorinated pyridazine-3-ones intended to be used in the treatment of broncho-pulmonary diseases, said compound, or one of its pharmaceutically acceptable salts, having the following formula (1):

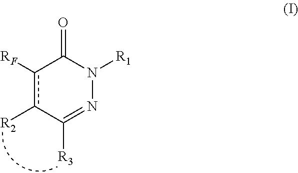

Wherein:
R1 represents H, and alkyl, an aryl or a heteroaryl,
R2 and R3 represents either independently of each other, and H, an alkyl, an aryle or a heteroaryl, or R2 and R3 are bridged within a same ring or via several rings;
R5 represents CF₃, (CF₂)ₙCF₃ or CF₂H with “n” representing an integer comprised between 1 and 7.

Advantageously, the compound of the invention or a pharmaceutical acceptable salt of said compound, intended to be used in the treatment of broncho-pulmonary diseases has a formula wherein:
R1 represents H, a linear or branched C₁-C₁₀ alkyl, or an aryl selected from a man the phenyl group C₆H₅, the tolyl group C₆H₄ CH₃, the xylyl group C₆H₃ (CH₃)₂, the naphthyl group C₁₀H₇, the 4-methoxyphenyl group C₆H₄OCH₃, the 3,4-dimethoxyphenyl group C₆H₃ (OCH₃)₂, and the 4-N-heptyloxyphenol) C₆H₄O (CH₂)₆ CH₃;
all R2 and R3 represent, independently of each other, H, A linear and/or functionalised C₁-C₁₀ alkyl, and aryl selected from among the phenyl group C₆H₅, tolyl group C₆H₄ CH₃, the xylyl group C₆H₃ (CH₃)₂, the naphthyl group C₁₀H₇, or a heteroaryl selected from among pyridyl, pyridazynyl, pyrimidyl, triazinyl, pyrrolyl, pyrazolyl, imidazolyl, (1,2,3) and (1,2,4)-triazolyl, pyrazinyl, pyrimidinyl, tetrazolyl, furyl, thienyl, isoxazole, thiazolyl, phenyl and oxazolyl, or R2 and R3 are bridged within a same ring with 5 or 6 carbon atoms;
RF represents CF₃, (CF₂)_{N} CF₃ of CF₂H with N representing an integer comprise between 1 and 7.

According to a particular involvement of involvement of the invention, the compound or a pharmaceutically acceptable salt of sed compound, intended to be used in the treatment of obstructive pulmonary diseases, is selected from among the list of the following compounds:
N2-methyl-4-(trifluromethyl)-6-(4′-methoxyphenyl)-4,5-dihydropyridazin-3 (2H)-one
4-(trifluoromethyl)-6-(3′ 4′-dimethoxyphenyl) pyridazine-3 (2H)-one
6-(4′-difluoromethoxy) phenyl)-4-(trifluoromethyl), 4,5-dihydropyridazin-3, (2H)-one,
6-(4′-difluoromethoxy) phenyl)-4-(trifluromethyl), pyridazine-3 (2H)-one,
2-phenyl-6-(P-tolyl)-4-) (trifluromethyl)-4,5-, dihydropyridazin-3 (2H)-one,
4-(trifluoromethyl)-2-phenyl-6-(P-tolyl) pyridazine-3 (2H)-one
6-(4′-(dimethoxyphenyl)-3′-methoxy-phenyl)-4-(trifluoromethyl)-4,5-dihydropyridazin-3 (2H)-one.
6-(4′-(difluoromethoxy-3′-methoxyphenyl)-4-(trifluoromethyl), pyridazine-3 (2H)-one.

According to a particular involvement of the invention, the compound or a pharmaceutically acceptable salt of said compound, having a formula wherein:
R1 represents H
R2 represents H
R3 represents the 3, 4-dimethoxyphenyl aryl:

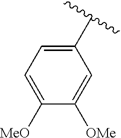 [SMILES: *c1ccc(OC)c(OC)c1]

RF represents CF3.

Said compound having the formula (Ic):

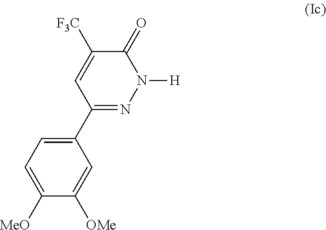 [SMILES: COc1ccc(-c2cc(C(F)(F)F)c(=O)[nH]n2)cc1OC]

According to another embodiment of the invention, the compound, or a pharmaceutically acceptable salt of said compound, having a formula wherein:
R1 represents CH3.
R2 represents H.
R3 represents the following aryl:

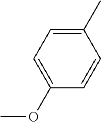

RF represents a CF3.

compound having the formula (Ib) below:

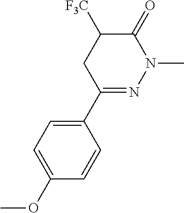

According to another particularity, the compound of the invention or one of its pharmaceutically acceptable source is a compound:
Intended to be used as an inhibitor drug of phosphodiesterases of type IV,
Intended to be used to the treatment of obstructive chronic pneumopathies.
Intended to be used in the treatment of asthma.
Intended to be used in the treatment of cystic fibrosis.
Intended to be used as an active ingredient of a drug, against chronic obstructive pulmonary diseases.

The present invention also relates to the use of a compound of the invention, for obtaining a drug intended for therapeutic use for chronic obstructive lung diseases.

The present invention includes many advantages. On the one hand, the compound according to the invention gives the possibility of efficiently treating lung affections, like OCPM, and more particularly OCPB, cystic fibrosis and asthma. On the other hand, the compounds according to invention have lower toxicity and reduced secondary effects, as compared with the compound which are proposed in the state of the art.

Indeed, the compounds according to the invention give the possibility of targeting, in a particularly specific way, the enzyme phosphodiesterase of type IV (PDE4), the latter forming a therapeutic target validated in the treatment of lung pathologies.

More particularly, the compounds according to the invention are particularly selective towards this target PDE4, as compared with other isoform of the enzyme, like the phosphodiesterases of type I (PDE1). In other words, the compound according to the invention mainly target the enzymes PDE4.

This particular selectivity gives the possibility of limiting the secondary or undesirable effects and the toxicity which are associated with the usual treatments. Notably, the secondary effects may appear by diarrhea, a loss of weight, nauseas, headaches or further signs of anxiety and of depression.

Finally, another advantage of the compounds according to the invention lies in the fact that their synthesis route is highly flexible. This advantageously gives the possibility of obtaining compounds having different points of structural variations, i.e. with different groups, thus providing access to many pharmaco-modulations, and thus giving the possibility of modulating the structure-activity-selectivity relationships.

Other features and advantages of the invention will become apparent from the detailed description which follows of non-limiting embodiments of the invention.

### BRIEF DESCRIPTION OF THE SEVERAL VIEWS OF THE DRAWINGS

Reference will be made to the appended figures which represent the compounds known in the state of the art for treating chronic obstructive pulmonary diseases and the compounds according to the invention.

FIG. 1A represents the chemical formula of zardaverine and FIG. 1B represents the chemical formula of an analog of zardaverine, these compounds being known in the state of the art.

FIG. 2A represents the general chemical formula (I) of the compound belonging to the family of fluorinated pyridazin-3-ones according to the present invention.

FIG. 2B represents the chemical formula (Ia) corresponding to an embodiment of the compound (I) according to the invention, the latter then consisting in a fluorinated bicyclic pyridazin-3-one.

FIG. 2C represents the chemical formula (Ib).

FIG. 2D represents the corresponding chemical formula (Ic) each to another particular embodiments of the compound according to the invention.

FIG. 3 schematically illustrates a particular embodiment of a method giving the possibility of resulting in a compound (I) according to the present invention, and notably the initial compounds, the intermediate products obtained during the reaction and the reaction conditions.

FIG. 4 illustrates an example of a method for obtaining the compound (Ib), and notably the initial compounds, the intermediate products obtained during the reaction and the reaction conditions.

FIG. 5 illustrates an example of a method for obtaining the compound (Ic), and notably the initial compounds, the intermediate products obtained during the reaction and the reaction conditions.

### DETAILED DESCRIPTION OF THE INVENTION

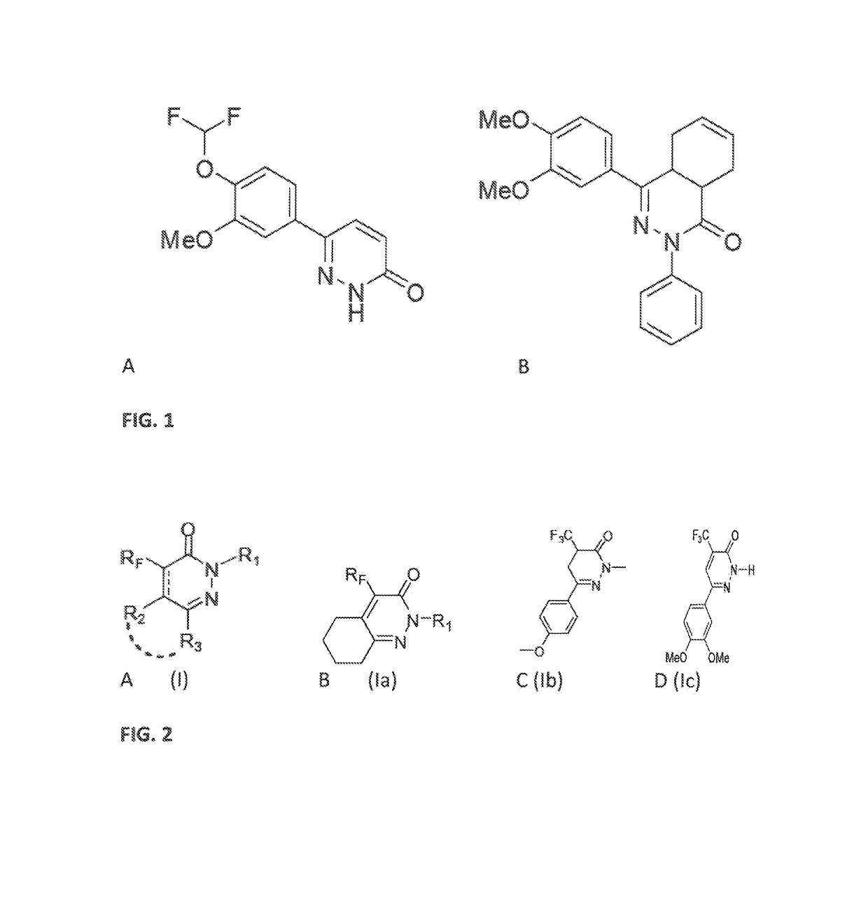

The compounds of the state of the art have already been mentioned above, notably zardaverine, for which the structure is visible in FIG. 1A, and the analog of the latter, represented in FIG. 1B.

These compounds in particular have a pyridazin-3-one unit of chemical formula C₄H₄N₂O or preferably a 4,5-dihydropyridazin-3-one unit of chemical formula C₄H₆N₂O including, on the one hand, a pyridazine ring (or 1,2-diazine) consisting in a heterocyclic dinitrogen-containing molecule of chemical formula C₄H₄N₂, therefore including two nitrogen atoms (N) in positions 1 and 2 in an aromatic ring with six members and, on the other hand, a carbonyl function in position 3 on the ring, in other words one has a C═O group in position 3 on the ring.

However, the inhibitor activity of these compounds towards the PDE4 enzyme further remains to be improved, as well as the selectivity of the compounds towards said enzyme PDE4.

Indeed, the compounds with a pyridazin-3-one unit act on PDE4, inhibiting its operation.

The enzyme PDE4 belongs to the large family of phosphodiestrases, and cataylyzes the transformation of AMPc (cyclic 3′5′adenosine mono-phosphate), which is the active form, in AMP, which is the inactive form, by hydrolyzing the phosphate bond present in position 3′ on the AMPc.

The phosphodiesterases are classified in 11 different families. Because of the large number of isoenzymes phosphodiestrases, the compounds with a pyridazin-3-one unit may interact with enzymes other than PDE4, like for example PDE1.

Now, PDE4 is the major enzyme of metabolism of AMPc, this enzyme notably intervenes in cells involved in the inflammatory response and the immune cells. The PDE4s therefore are a preferred target and the inhibitors of PDE4 have a very interesting potential for treating the inflammatory response related to broncho-pulmonary pathologies like asthma, COPMs, cystic fibrosis, etc.

Thus, the inhibitors of PDE4 suppress the release of cytokines and of other inflammatory molecules.

Accordingly, it is of particular interest to develop compounds which inhibit, in a highly selective way, the PDE4s, by avoiding action on other families of phosphodiesterases.

A compound belonging to the family of pyridazin-3-ones was therefore developed, notably fluorinated pyridazin-3-ones i.e. including at least one fluorine atom, for its use in the treatment of obstructive pulmonary diseases, and having the general formula (I) illustrated below and in the appended FIG. 2:

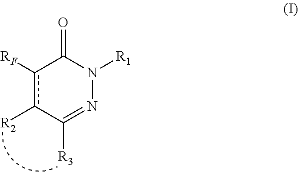

In this compound of formula (I), the identified radical R₁ represents H, an alkyl, an aryl or a heteroaryl.

As regards the radicals R₂ and R₃, the latter may represent independently of each other, H, an alkyl, an aryl or a heteroaryl.

In the case when the dotted line bond connecting the carbon atoms C4 and C5 represent a saturated bond, the compound of the invention belongs to the particular family of fluorinated 4,5-dihydropyridazin-3-one.

The radicals R₂ and R₃ may also be bridged within a same ring or via several rings, which is illustrated by the dotted lines connecting R₂ and R₃ in the above formula (I). The dotted lines of formula (I) between the radicals R₂ and R₃ represent the possible ring(s) structurally connecting the chemical formula of the invention.

Now as regards the radical R_{F}, the latter preferably includes at least two fluorine atoms and may represent CF₃, (CF₂)ₙCF₃ or CF₂H with n representing an integer comprised between 1 and 7 in (CF₂)ₙCF₃.

The fluorinated unit in position 4 on the compound of formula I according to the invention advantageously gives the possibility of increasing the selectivity of said compound towards PDE4.

From this increased specificity towards PDE4s there results a limitation of the potential undesirable or secondary effects which may result from the taking of these compounds.

Indeed, a chemical compound inhibiting phosphodiesterases other than the PDE4s may notably cause nauseas, headaches, diarrheas, loss of weight or further signs of anxiety and depression, which may be very unpleasant, or even dangerous for the patient.

The compound of formula (I) according to the invention therefore gives the possibility of improving the life quality of the patients, by limiting the secondary effects related to the taking of this type of molecule.

Further, from this selectivity there results that the compound of formula (I) is particularly efficient in the treatment of obstructive pulmonary pathologies, i.e. pathologies affecting the bronchial tubes and/or the lungs.

Said compound thus causes a reduction in the outbreaks of the disease, like coughing, breathlessness, production of mucus, etc. The deterioration of the respiratory function is then avoided, as well as hospitalization of the patient.

The compound of formula (I) was mentioned above. However, this is not a limiting embodiment of the invention, and protection is also sought for a pharmaceutically acceptable salt of said compound of formula (I).

Herein, the term of <<pharmaceutically acceptable salt>> relates to a salt not having any toxicity, irritation, allergic response or other effects having a hazardous effect for the health of the patient.

The salt of the present compound of formula (I) according to the invention may be obtained by subjecting said compound to an ordinary reaction of salt formation.

The salt of the present compound of formula (I) may for example be an ammonium salt or a metal salt, such as alkaline metal salt, for example a sodium or potassium salt, or an earth-alkaline metal salt, for example a calcium or magnesium salt.

By the term of <<alkyl>> is meant a hydrocarbon radical with a linear or branched chain with an unsaturated bond, which may be functionalized. In other words, the carbon chain may bear one or several chemical functions or <<functional groups>>.

For the compound (I) according to the invention, advantageous examples of alkyl groups include, without being limited thereto, groups comprising from 1 to 10 carbon atoms (C1 to C10), either linear or branched which may be either functionalized or not. Still more preferentially, this is a lower C₁-C₄ alkyl.

By the term of <<aryl>> is meant a functional group derived from an aromatic hydrocarbon, generally a phenyl (C6) or naphthyl (bicyclic C10) group and optionally substituted with at least one and up to three group(s) or atom(s) selected from the assembly formed by at least the alkyl, alkyloxy or alkoxy groups (an alkyl group bound to an oxygen group, —O—R), halogen (F, Cl, Br or I) or nitro (—NO₂), alkylthio (—RS), cyano (CN), hydroxyl (—OH), amine (—NH₂), alkylamine (—RNH), dialkylamine (—NR₂), carbonyl (—C═O), ketone (—COR), ester (—CO₂R), amide (—CONRR′).

Examples of aryl groups include, without being limited thereto, the phenyl groups C₆H₅, the tolyl group C₆H₄CH₃, the xylyl group C₆H₃(CH₃)₂, the naphthyl group C₁₀H₇, the 4-methoxyphenyl group C₆H₄OCH₃, the 3,4-dimethoxyphenyl group C₆H₃(OCH₃)₂, and the 4-(n-heptyloxyphenyl) group C₆H₄O(CH₂)₆CH₃.

Preferably, these aryl groups may be substituted with at least an up to three groups, notably alkyl, carbonyl or alkyloxy or another one as indicated above.

By the term of <<heteroaryl>> is meant a monocylic or polycyclic aromatic ring comprising in the ring carbon (C) and hydrogen (H) atoms, one or several heteroatoms selected independently from notably nitrogen (N), oxygen (O) and sulphur (S), phosphorus (P), and which may have a substitution scheme as described earlier with the term of <<aryl>>.

By <<heteroatom>>, is meant an atom of an organic molecule having at least one electron doublet but which is neither carbon nor hydrogen, and not a metal. The most frequent heteroatoms are oxygen, nitrogen, sulphur, phosphorus and halogens, like fluorine (F), bromine (Br), chlorine (Cl) and iodine (I).

Examples illustrating heteroaryl groups include, without however being limited thereto, groups such as pyridinyl, pyridazinyl, pyrimidyl, triazinyl, pyrrolyl, pyrazolyl, imidazolyl, (1,2,3)- and (1,2,4)-triazolyl, pyrazinyl, pyrimidinyl, tetrazolyl, furyl, thienyl, isoxazolyl, thiazolyl and oxazolyl.

Advantageously, when one of both groups R₂ or R₃ consist in an aryl group or in a heteroaryl group, the second group R₂ or R₃ consist in an alkyl C₁-C₁₀ group, preferably C₁-C₄ group, or in a hydrogen atom H.

It has also been mentioned that R₂ and R₃ may be bridged within a same ring or via several rings.

Preferably, when R₂ and R₃ are bridged, they are bridged via a single homocylic ring only having carbon atoms C and hydrogen atoms H, or via a single heterocyclic ring having at least one atom different from C, H atoms in the ring.

Advantageously, the bridging between R₂ and R₃ is achieved via a single ring with saturated bonds, having 5 or 6 atoms, preferably a saturated ring with 6 carbon atoms.

Thus, the compound according to the invention may for example have the formula (Ia) below:

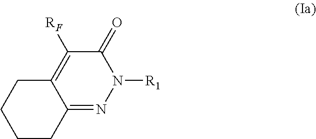

The compound (Ia) then consists in a fluorinated bicyclic pyridazin-3-one with:
R₁ represents H, an alkyl, an aryl or a heteroaryl.
R_{F} represents CF₃, (CF₂)n CF₃ or CF₂H with n representing an integer comprised between 1 and 7.

According to a particular embodiment, the compound has a formula (I) wherein:
R₁ represents H or a lower C₁-C₄ alkyl
R₂ represents H or a lower C₁-C₄ alkyl
R₃ represents an aryl substituted with a lower alkyl C₁-C₄ group or with one or several alkoxy groups.
R_{F} represents CF₃ (CF₂)n CF₃ or CF₂H with n representing an integer comprised between 1 and 3.

In a still further advantageous embodiment of the invention, the compound, or a pharmaceutically acceptable salt of said compound, has a formula (Ib) wherein:
R₁ represents CH₃
R₂ represents H
R₃ represents the following aryl or p-methoxyphenyl:

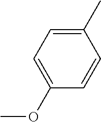

R_{F} represents CF3.

This formula corresponds to the formula (Ib) above:

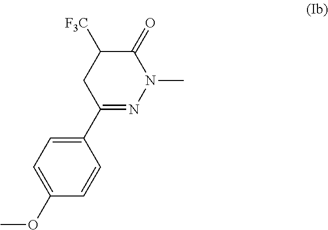

The formula (Ib) corresponds to N₂-methyl-4-(trifluoromethyl)-6-(4′-methoxyphenyl)-4,5-dihydroxypyridazin-3(2H)-one.

In a still further advantageous embodiment of the invention, the compound, or a pharmaceutically acceptable salt of said compound, has a formula (Ic) wherein:
R₁ represents H
R₂ represents H
R₃ represents the following aryl or 3,4-dimethoxyphenyl:

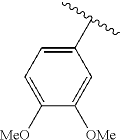 [SMILES: *c1ccc(OC)c(OC)c1]

R_{F} represents CF₃.

This formula corresponds to the formula (Ib) above:

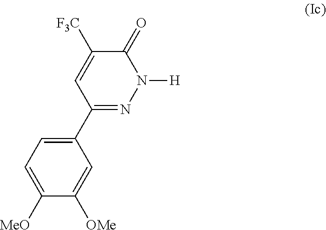 [SMILES: COc1ccc(-c2cc(C(F)(F)F)c(=O)[nH]n2)cc1OC]

This formula corresponds to the formula (Ic) corresponds to 4-(trifluoromethyl)-6-(3′,4′-dimethoxyphenyl)pyridazin-3(2H)-one.

Other particularly preferred compounds of the invention are:
6-(4′-(difluoromethoxy)phenyl)-4-(trifluoromethyl)-4,5-dihydropyridazin-3(2H)-one (Id)
6-(4′-(difluoromethoxy)phenyl-4-(trifluoromethyl)pyradazin-3(2H)-one (Ie)
2-phenyl-6-(p-tolyl)-4-(trifluoromethyl)-4,5-dihydropyridazin-3(2H)-one (If)
4-(trifluoromethyl)-2-phenyl-6-(p-tolyl)pyradizin-3(2H)-one (Ig)
6-(4′-(difluoromethoxy)-3′-methoxy-phenyl)-4-(trifluoromethyl)-4,5-dihydropyridazin-3(2H)-one (Ih)
6-(4′-(difluoromethoxy)-3′-methoxy-phenyl)-4-(trifluoromethyl)pyridazin-3(2H)-one (Ii)

These preferred compounds above have the advantage of having large selectivity towards PDE4 and are therefore particularly efficient in the treatment of pulmonary diseases.

The compound of formula (I) according to the invention, as well as the compound of formula (Ic), may advantageously be obtained by the method schematically illustrated in the enclosed FIG. 3.

One starts from a fluorinated ketene dithioketal compound of formula (III) with R_{F} corresponding to CF₃, (CF₂)ₙCF₃ or CF₂H with n representing an integer comprised between 1 and 7.

This compound is reactive with a potassium enolate of formula (IV), in the presence of tetrahydrofurane (THF) as a solvent, at a temperature comprised between 0 and 25° C. for a period of the order of 4-10 h.

Thus the intermediate of formula (V) corresponding to a perfluorinated ketene dithioketal compound illustrated in FIG. 3 is thereby obtained.

This intermediate of formula (V) is then subject to an acid hydrolysis reaction in the presence of trifluoroacetic acid and water during refluxing with heating. This acid hydrolysis reaction gives the possibility of obtaining a second intermediate of formula (VI).

The intermediate compound (VI) then undergoes a condensation reaction with hydrazine (R₁NHNH₂). The condensation reaction is carried out in the presence of para-toluenesulfonic acid (PTSA) in a solvent, in a device for refluxing with heating, for a duration of the order of 1 to 5 hours.

Preferably, this solvent consists in toluene or in glacial acetic acid (AcOH). The condensation reaction gives the possibility of obtaining the compound (I).

After cooling, the compound (I) according to the invention is then purified. The purification of said compound (I) is either carried out by chromatography on silica gel, advantageously in the presence of a mixture of petroleum ether and of ethyl acetate, or by precipitation of the product (I) by adding water.

In order to obtain the compound Ic, the compound (I) should then undergo an oxidation reaction in the presence of copper chloride (CuCl₂) in acetonitrile in reflux, for about 4 h.

The nature of the fluorinated unit, which may notably consist in a group CF₃ or (CF₂)ₙCF₃ or CF₂H, derived from the fluorinated ketene dithioketal compound (III) gives the possibility, in a particularly interesting way, of modulating the selectivity of the compound (I) according to the invention against the enzyme PDE4.

Accordingly, when said compound (I) will be administered to a patient for treating a broncho-pulmonary pathology, the potential secondary effects of the compound (I) will be limited, notably as compared with therapeutic molecules already used in the state of the art for treating this type of pathologies.

The unit R₁ depends on the hydrazine molecule (R₁NHNH₂) used during the condensation step. This unit R₁ gives the possibility of adapting, according to its structure, the interactions with the active site of the PDE enzymes.

The units R₂ and R₃ are related to the use of different ketones, corresponding to the compound (IV) of FIG. 3, during the preparation of the fluorinated dithioketal intermediate (V). The possibility of a system wherein the units R₂ and R₃ are bridged in order to lead to a bicyclic pyridazin-3-one molecule with 5 or 6 members, notably with 6 members as illustrated in the appended FIG. 2B, was already mentioned.

Definitively, because of the highly flexible synthesis method of the compound (I) according to the invention, illustrated in FIG. 3, each of the different units, R₁, R_{F}, R₂ and R₃, form a point for structural variation. This provides accordingly access to many pharmaco-modulations of said compound (I) for obtaining an optimal relationship between the structure, the activity and the selectivity of the compound (I) relatively to PDE4, so that said compound (I) is efficient in the treatment of broncho-pulmonary diseases.

In particular, the compound according to the invention, whether this is notably the compound (I), the compound (Ia) or the compound (Ib), or a pharmaceutically acceptable salt of one of these compounds, is used for inhibiting the enzymes phosphodiesterases of type IV.

In a preferential example, the compound according to the invention, whether this is the compound (I), (Ia) or (Ib) notably is used in the treatment of obstructive chronic obstructive pulmonary diseases (OCBP).

In another exemplary embodiment, said compounds according to the invention which were mentioned above are used for treating asthma.

The examples below, non-limiting of the invention, illustrate on the one hand the preparation of compounds according to the invention, notably the compounds having the formula (Ic), 4-(trifluoromethyl)-6-(3,4-dimethoxyphenyl)pyridazin-3(2H)-one, (Ig), 4-(trifluoromethyl)-2-phenyl-6-(p-tolyl)pyradizin-3(2H)-one and (Ii), 6-(4′-(difluoromethoxy)-3′-methoxy-phenyl)-4-(trifluoromethyl)pyridazin-3(2H)-one and, on the other hand the activity and interest for these compounds.

### Example 1: Preparation of N2-methyl-4-(trifluoromethyl)-6-(4′-methoxyphenyl)-4,5-dihydropyridazin-3(2H)-one of formula (Ib)

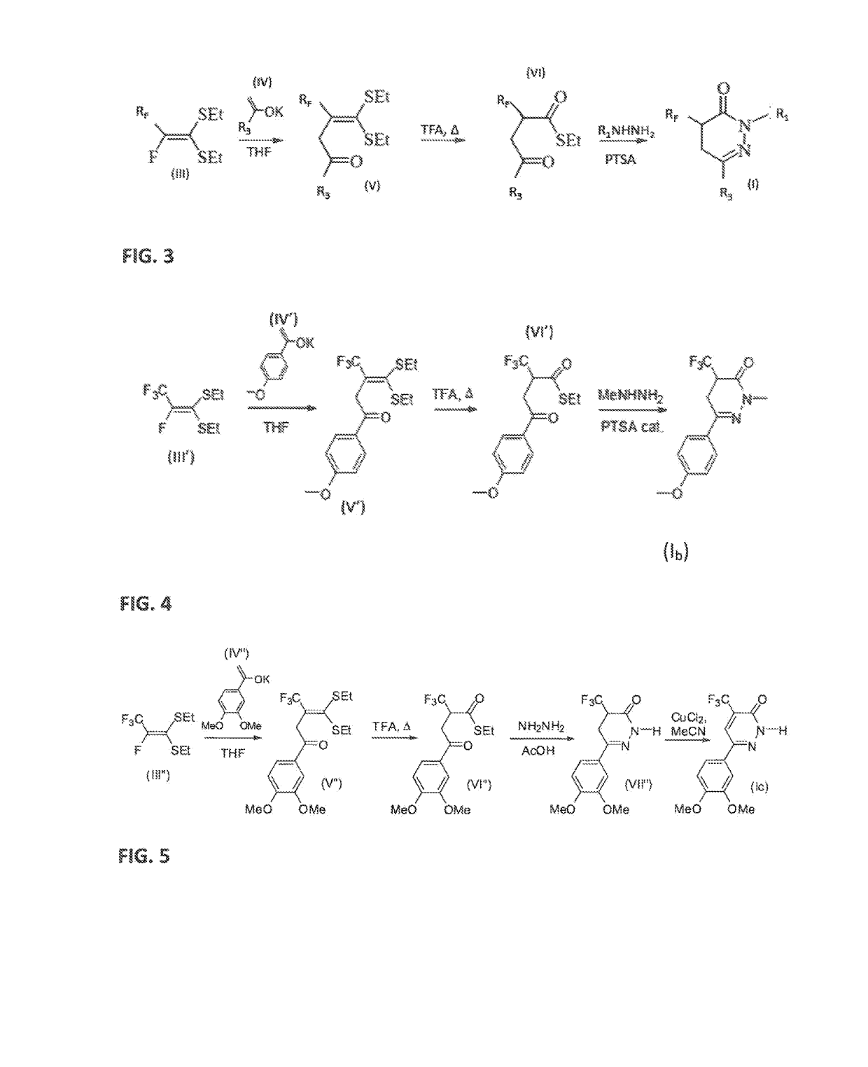

In this example, reference is made to the appended FIG. 4, which illustrates the reaction which gives the possibility of obtaining the compound of formula (Ib).

A solution of potassium hydride and a solution of 4-methoxyacetophenone is mixed preferably in an argon atmosphere at 0° C., in the presence of a solvent, preferably tetrahydrofurane (THF), this forms the mixture 1.

To said mixture 1, is added after 10 to 20 mins of stirring, preferably 15 mins, a solution of perfluoroketene dithioacetal solution of formula (III′), this forms the mixture 2.

Said mixture 2 is stirred, at room temperature for 2 h 45 to 3 h 30, preferably 3 h.

The current reaction is hydrolyzed occurring within the mixture 2 with water.

The aqueous phase of the mixture 2, is extracted notably with ether.

The organic phase of the mixture 2 is dried, preferably on magnesium sulfate.

The organic phase of the mixture 2 is filtered and evaporated, preferably under reduced pressure.

A chromatography on a column is carried out for obtaining, advantageously as an oil, the compound (V′) of formula C₁₆H₁₉F₃O₂S₂ called 1,1-Bis(ethylsulfanyl)-4-(4′-methoxyphenyl)-2-trifluoromethyl-but-1-ene-4-one and, preferably a silica column is used,

Said compound (V′) is mixed with water and with trifluoroacetic acid (TFA), this forms the mixture 3.

The mixture 3 is refluxed, for a period of the order of 10 h.

After cooling, the mixture 3 is neutralized with a saturated aqueous solution, preferably with NaHCO₃.

The aqueous phase of the mixture 3, is extracted preferably with methylene chloride.

The organic phases of the mixture 3 are dried, filtered and evaporated.

A column chromatography is carried out in order to obtain, advantageously as an oil, the compound (VI′) of formula C₁₄H₁₅F₃O₃S called S-ethyl 4-(4′-methoxyphenyl)-2-trifluoromethyl-4-oxo-butanethioate, preferably a silica column is used.

Said compound (VI′) is mixed with glacial acetic acid and methyl hydrazine, the latter forms the mixture 4.

The mixture 4 is refluxed, preferably for a period of the order of 1 h.

After cooling the mixture 4, the product (Ib) present in the mixture 4 is precipitated with water.

After filtration, washing and drying in vacuo, preferably at a temperature of 100° C. for a period of the order of 16 h, the product (Ib) is obtained, pure in a solid form, of formula C₁₃H₁₃F₃N₂O₂ called N2-methyl-4-(trifluoromethyl)-6-(4′-methoxyphenyl)-4,5-dihydropyridazin-3(2H)-one.

### Example 2: Preparation of 4-(trifluoromethyl)-6-(3′,4′-dimethoxyphenyl)pyridazin-3(2H)-one of formula (Ic)

In this example, reference is made to the appended FIG. 5, which illustrates the reaction which gives the possibility of obtaining the compound of formula (Ic).

A solution of potassium hydride and a solution of 3,4-dimethoxyacetophenone is mixed preferably under an argon atmosphere at 0° C., in the presence of a solvent, preferably tetrahydrofurane (THF), this forms the mixture 1.

To said mixture 1, after 10 to 20 mins of stirring, preferably 15 mins, is added a solution of perfluoroketene dithioacetal of formula (III″), this forms the mixture 2.

Said mixture 2 is stirred, at room temperature for 2 h 45 to 3 h 30, preferably 3 h.

The current reaction occurring within the mixture 2 is hydrolyzed with water.

The aqueous phase of the mixture 2 is extracted, notably with ether.

The organic phase of the mixture 2 is dried, preferably on magnesium sulfate.

The organic phase of the mixture 2 is filtered and evaporated, preferably under reduced pressure.

A chromatography on a column is carried out for obtaining, advantageously as an oil, the compound (V″) of formula C₁₇H₂₁F₃O₃S₂ called 1,1-Bis(ethylsulfanyl)-4-(3′,4′-dimethoxyphenyl)-2-trifluoromethyl-but-1-ene-4-one and preferably a silica column is used.

Said compound (V″) is mixed with water and trifluoroacetic acid (TFA), this forms the mixture 3.

The mixture 3 is refluxed, for a period of the order of 10 h.

After cooling, the mixture 3 is neutralized with a saturated aqueous solution, preferably with NaHCO₃.

The aqueous phase of the mixture 3 is extracted, preferably with methylene chloride.

The organic phases of the mixture 3 are dried, filtered and evaporated.

A chromatography on a column is carried out for obtaining, advantageously as an oil, the compound (VI″) of formula C₁₅H₁₇F₃O₄S called S-ethyl 4-(3′,4′-dimethoxyphenyl)-2-trifluoromethyl-4-oxo-butanethioate, preferably a silica column is used.

Said compound (VI″) is mixed with glacial acetic acid and hydrazine hydrate, this forms the mixture 4.

The mixture 4 is refluxed, preferably for a period of the order of 1 h.

After cooling the mixture 4, the product (VII′) present in the mixture 4 is precipitated.

After filtration, washing and drying in vacuo, preferably at a temperature of 100° C. for a period of the order of 16 h, the product (VII″) is obtained pure in a solid form, of formula C₁₃H₁₃F₃N₂O₃ called 6-(3′,4′-dimethoxyphenyl)-4-trifluoromethyl-4,5-dihydropyridazin-3(2H)-one.

The compound (VII″) is mixed under an argon atmosphere in anhydrous acetonitrile with copper chloride, this forming the mixture 5.

The mixture 5 is refluxed, preferably for a period of the order of 4 h.

After cooling, the mixture 5 is purified by column chromatography in order to obtain the compound (Ic), in the form of a solid, of formula C₁₃H₁₃F₃N₂O₃ called 4-(trifluoromethyl)-6-(3′,4′-dimethoxyphenyl)pyridazin-3(2H)-one.

The whole of these steps gives the possibility of obtaining the compound (Ib) and (Ic) according to the invention.

### Example 3: Preparation of 6-(4′-(difluromethoxy)phenyl)-4-(trifluoromethyl)-4,5-dihydropyridazin-3(2H)-one (Id)

In this example, reference is made to the compound, having relatively to the structure of the general compound (I), R₁=H, R_{F}=CF₃, R₂=H, R₃=4-(difluoromethoxy)phenyl.

A potassium hydride solution and a solution of 4-(difluoromethoxy)acetophenone is mixed, preferably under an argon atmosphere at 0° C., in the presence of a solvent, preferably tetrahydrofurane (THF), this forms the mixture 1.

To said mixture 1, after 10 to 20 mins of stirring, preferably 15 mins, is added a solution of perfluoroketene dithioacetal of formula (III′), this forms the mixture 2.

Said mixture 2 is stirred, at room temperature for 2 h 45 to 3 h 30, preferably 3 h.

The current reaction occurring within the mixture 2 is hydrolyzed with water.

The aqueous phase of the mixture 2 is notably extracted with ether.

The organic phase of the mixture 2 is preferably dried on magnesium sulfate.

The organic phase of the mixture 2 is filtered and evaporated, preferably under reduced pressure.

A chromatography on a column is carried out in order to obtain, advantageously as an oil, the compound (V′″) of formula C₁₈H₁₇F₅O₂S₂ called 1,1-Bis(ethylsulfanyl)-4-(4′-(difluoromethoxy)phenyl)-2-trifluoromethyl-but-1-ene-4-one and, a silica column is preferably used.

Said compound (V′″) is mixed with water and trifluoroacetic acid (TFA), this forms the mixture 3.

The mixture 3 is refluxed, for a period of the order of 10 h.

After cooling, the mixture 3 is neutralized with a saturated aqueous solution, preferably with NaHCO₃.

The aqueous phase of the mixture 3 is preferably extracted with methylene chloride.

The organic phases of the mixture 3 are dried, filtered and evaporated.

A chromatography on a column is carried out for obtaining, advantageously as an oil, the compound (VI′″) of formula C₁₄H₁₃F₅O₃S called S-ethyl 4-(4′-(difluoromethoxy)phenyl)-2-trifluoromethyl-4-oxo-butanethioate, preferably a silica column is used.

Said compound (VI′″) is mixed with glacial acetic acid and hydrazine hydrate, this forms the mixture 4.

The mixture 4 is refluxed, preferably for a period of the order of 1 h.

After cooling of the mixture 4, the product (Id) present in the mixture 4 is precipitated with water.

After filtration, washing and drying in vacuo, preferably at a temperature of 100° C. for a period of the order of 16 h, the product (Id) is obtained pure as a solid, of formula C₁₂H₉F₅N₂O₂ called 6-(4′-(difluoromethoxy)phenyl)-4-(trifluoromethyl-4,5-dihydropyridazin-3(2H)-one.

### Example 4: Preparation of 6-(4′-(difluoromethoxy)phenyl)-4-(trifluoromethyl)pyridazin-3(2H)-one of formula (Ie)

In this example, reference is made to the compound, having relatively to the structure of the general compound (I), R₁=H, R_{F}=CF₃, R₂=H, R₃=4-(difluoromethoxy)phenyl.

The compound (Id) is mixed in anhydrous acetonitrile under an argon atmosphere with copper chloride, this forms the mixture 5.

The mixture 5 is refluxed, preferably for a period of the order of 4 h.

After cooling, the mixture 5 is purified by column chromatography in order to obtain the compound (Ie), in the form of a solid, of formula C₁₂H₇F₅N₂O₂ called 6-(4′-(difluoromethoxy)phenyl)-4-(trifluoromethyl)pyridazin-3(2H)-one.

### Example 5: Preparation of 2-phenyl-6-(p-tolyl)-4-(trifluoromethyl)-4,5-dihydropyridazin-3(2H)-one (If)

In this example, reference is made to the compound, having relatively to the structure of the general compound (I), R₁=phenyl, R_{F}=CF₃, R₂=H, R₃=p-tolyl.

A solution of potassium hydride and a solution of 1-(p-tolyl)ethanone is mixed, preferably under an argon atmosphere at 0° C., in the presence of a solvent, preferably tetrahydrofurane (THF), this forms the mixture 1.

To said mixture 1, after 10 to 20 mins of stirring, preferably 15 mins, is added a solution of perfluoroketene dithioacetal of formula (III′), this forms the mixture 2.

Said mixture 2 is stirred at room temperature for 2 h 45 to 3 h 30, preferably 3 h.

The current reaction occurring within the mixture 2 is hydrolyzed with water.

The aqueous phase of the mixture 2, is extracted notably with ether.

The organic phase of the mixture 2 is dried preferably on magnesium sulphate.

The organic phase of the mixture 2 is filtered and evaporated, preferably under reduced pressure.

A chromatography on a column is carried out in order to obtain, advantageously as an oil, the compound (V″″) of formula C₁₆H₁₉F₃OS₂ called 1,1-Bis(ethylsulfanyl)-2-trifluoromethyl-4-(p-tolyl)-but-1-ene-4-one and preferably a silica column is used.

Said compound (V″″) is mixed with water and trifluoroacetic acid (TFA), this forms the mixture 3.

The mixture 3 is refluxed, for a period of the order of 10 h.

After cooling, the mixture 3 is neutralized with a saturated aqueous solution, preferably with NaHCO₃.

The aqueous phase of the mixture 3 is preferably extracted with methylene chloride.

The organic phases of the mixture 3 are dried, filtered and evaporated.

A chromatography on a column is carried out in order to obtain, advantageously as an oil, the compound (VI″″) of formula C₁₄H₁₅F₃O₂S called S-ethyl 2-trifluoromethyl-4-(p-tolyl)-4-oxo-butanethioate, preferably a silica column is used.

Said compound (VI″″) is mixed with glacial acetic acid and phenylhydrazine, this forms the mixture 4.

The mixture 4 is refluxed, preferably for a period of the order of 1 h.

After cooling the mixture 4, the product (If) present in the mixture 4 is precipitated with water.

After filtration, washing and drying in vacuo, preferably at a temperature of 100° C. for a period of the order of 16 h, the product (If) is obtained pure in a solid form, with formula C₁₈H₁₅F₃N₂O called 2-phenyl-6-(p-tolyl)-4-(trifluoromethyl)-4,5-dihydropyridazin-3(2H)-one.

### Example 6: Preparation of 4-(trifluoromethyl)-2-phenyl-6-(p-tolyl)pyridazin-3(2H)-one (Ig)

In this example, reference is made to the compound, having relatively to the structure of the general compound (I), R₁=phenyl, R_{F}=CF₃, R₂=H, R₃=p-tolyl.

The compound (If) is mixed under an argon atmosphere in anhydrous acetonitrile with copper chloride, this forms the mixture 5.

The mixture 5 is refluxed, preferably for a period of the order of 4 h.

After cooling, the mixture 5 is purified by column chromatography in order to obtain the compound (Ig), as a solid, of formula C₁₈H₁₃F₃N₂O called 4-(trifluoromethyl)-2-phenyl-6-(p-tolyl)pyridazin-3(2H)-one.

### Example 7: Preparation of 6-(4′-(difluromethoxy)-3′-methoxy-phenyl)-4-(trifluoromethyl)-4,5-dihydropyridazin-3(2H)-one (Ih)

In this example, reference is made to the compound, having relatively to the structure of the general compound (I), R₁=H, R_{F}=CF₃, R₂=H, R₃=4-(difluoromethoxy)-3-methoxy-phenyl.

A solution of potassium hydride and a solution of 4-(difluoromethoxy)-3-methoxy-acetophenone is mixed preferably under an argon atmosphere at 0° C., in the presence of a solvent, preferably tetrahydrofurane (THF), this forms the mixture 1.

To said mixture 1, after 10 to 20 mins of stirring, preferably 15 mins, is added a solution of perfluoroketene dithioacetal of formula (III′), this forms the mixture 2.

Said mixture 2 is stirred at room temperature for 2 h 45 to 3 h 30, preferably 3 h.

The current reaction occurring within the mixture 2 is hydrolyzed with water.

The aqueous phase of the mixture 2, is notably extracted with ether.

The organic phase of the mixture 2 is preferably dried on magnesium sulphate.

The organic phase of the mixture 2 is filtered and evaporated preferably under reduced pressure.

A chromatography on a column is carried out in order to obtain, advantageously as an oil, the compound (V″″′) of formula C₁₇H₁₉F₅O₃S₂ called 1,1-Bis(ethylsulfanyl)-4-(4′-(difluoromethoxy)-3′-m ethoxy-phenyl)-2-trifluoromethyl-but-1-ene-4-one and preferably a silica column is used.

Said compound (V″″′) is mixed with water and trifluoroacetic acid (TFA), this forms the mixture 3.

The mixture 3 is refluxed, for a period of the order of 10 h.

After cooling, the mixture 3 is neutralized with a saturated aqueous solution, preferably with NaHCO₃.

The aqueous phase of the mixture 3 is preferably extracted with methylene chloride.

The organic phases of the mixture 3 are dried, filtered and evaporated.

A chromatography on a column is carried out for obtaining, advantageously as an oil, the compound (VI″″′) of formula C₁₅H₁₅F₅O₄S called S-ethyl 4-(4′-(difluoromethoxy)-3′-methoxy-phenyl)-2-trifluoromethyl-4-oxo-butanethioate, preferably a silica column is used.

Said compound (VI″″′) is mixed with glacial acetic acid and with hydrazine hydrate, this forms the mixture 4.

The mixture 4 is refluxed, preferably for a period of the order of 1 h.

After cooling of the mixture 4, the product (Ih) present in the mixture 4 is precipitated with water.

After filtration, washing and drying in vacuo, preferably at a temperature of 100° C. for a period of the order of 16 h, the product (Ih) is obtained pure as a solid, with formula C₁₃H₁₁F₅N₂O₃ called 6-(4′-(difluoromethoxy)-3′-methoxy-phenyl)-4-(trifluoromethyl-4,5-dihydropyridazin-3(2H)-one.

### Example 8: Preparation of 6-(4′-(difluoromethoxy)-3′-methoxy-phenyl)-4-(trifluoromethyl)pyridazin-3(2H)-one of formula (Ii)

In this example, reference is made to the compound, having relatively to the structure of the general compound (I), R₁=H, R_{F}=CF₃, R₂=H, R₃=4-(difluoromethoxy)-3-methoxy-phenyl.

The compound (Ih) is mixed under an argon atmosphere in the anhydrous acetonitrile with copper chloride, this forms the mixture 5.

The mixture 5 is refluxed, preferably for a period of the order of 4 h.

After cooling, the mixture 5 is purified by column chromatography in order to obtain the compound (Ii), as a solid, of formula C₁₃H₁₀F₅N₂O₃ called 6-(4′-(difluoromethoxy)-3′-methoxy-phenyl-4-(trifluoromethyl)pyridazin-3(2H)-one.

### Example 9: Evaluation of the action of 4-(trifluoromethyl)-6-(3′,4′-dimethoxyphenyl)-4,5-dihydropyridazin-3(2H)-one of formula (Ic), of 4-(trifluoromethyl)-2-phenyl-6-(p-tolyl)pyradizin-3(2H)-one (Ig) and of 6-(4′-(difluoromethoxy)-3′-methoxy-phenyl)-4-(trifluoromethyl)pyridazin-3(2H)-one of formula (Ii)

In order to evaluate the action of the compounds (Ic), (Ig) and (Ii) according to the invention, the activity of different sub-types of phosphodiesterases, either of type 4 or not, was tested in vitro by using a kit adapted to the recombinant isoforms of human phosphodiesterases PDE4B2 and PDE4D coded by Escherichia coli. The activity of the PDE1 and PDE10 enzymes was also monitored.

The principle of the test is based on the cleavage of AMPc by the enzymes phosphodiesterases. The nucleotide-5′ which is sorted out during the reaction is itself cleaved by an enzyme 5′-nucleotidase into a nucleoside and a phosphate, the latter being quantified by using the reagent Biomol Green™.

The PDE enzymes are incubated on microplates in the presence of AMPc, 5′-nucleotidase, in the presence of an inhibitor, more particularly corresponding to the compound (Ic), (Ig) or (Ii) according to the invention, or in the absence of any inhibitor (control), and this for a period of the order of 60 mins.

The reaction is stopped by adding an amount of 100 microliters of the reagent Biomol Green™ and the plate is incubated for further 30 mins in order to allow development of the colour before reading the absorbance by means of a micro-plate reader.

The compounds (Ic), (Ig) or (Ii) according to the invention are dissolved in dimethylsulfoxide (DMSO) so as to obtain a final DMSO concentration of 2%, this concentration not significantly affecting the activity of the PDE enzymes.

The study of the inhibition of the PDE enzymes by the compound (Ic), (Ig) or (Ii) according to the invention was carried out by testing five different concentrations (500 μM, 50 μM, 5 μM and 0.5 μM) of said compounds (Ic), (Ig) or (Ii).

The CI₅₀ values, for “inhibitory concentration 50”, corresponding to the amount, in μM, of compounds (Ic), (Ig) or (Ii) required for allowing inhibition of half of the activity of the PDE enzymes were then calculated by non-linear regression.

The CI₅₀s which were calculated represent the average value of three determinations which were achieved independently of each other.

A non-specific inhibitor of the PDE enzymes, 3-isobutyl-1-methylxanthin (IBMX) corresponds to the control.

The results gave the possibility of showing that the compound (Ic) has a CI₅₀ of 8.1 μM, that the compound (Ig) has a CI₅₀ of 15 μM and that the compound (Ii) has a CI₅₀ of 250 nM. As a comparison, the zardaverine molecule showed a CI₅₀ of 2 μM.

Further, it was also shown that said compounds (Ic), (Ig) or (Ii) are particularly selective against PDE4, relatively to PDE1. Indeed, the inhibition percentage of PDE4 by the compound (Ic) is 74% at a concentration of 50 μM of compound (Ic), 45% for (Ig) and 56% at a 5 μM concentration of compound (Ii). On the contrary, the compounds (Ic), (Ig) and (Ii) do not allow any inhibition of PDE1. The inhibition of PDE1 is actually 0% at a concentration of 50 μM of compound (Ic), (Ig) and (Ii). Further, the compound (Ii) has a selectivity towards PDE10 since it has a percentage of inhibition of 9% towards this enzyme at 50 μM.

The compounds (Ic), (Ig) or (Ii) therefore have on the one hand good inhibitory efficiency towards the therapeutic target, the enzyme PDE4, and, on the other hand increased specificity towards this target, relatively to other isoforms of the enzyme, notably PDE1 or PDE10.

Of course, the invention is not limited to the examples illustrated and described earlier which may have alternatives and modifications without however departing from the scope of the invention.

1. A compound being in a family of fluorinated pyridazin-3-ones, for treatment of broncho-pulmonary diseases, said compound, or one of its pharmaceutically acceptable salts, comprising a chemical composition having the following formula (I): 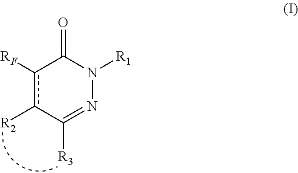
wherein:
R1 represents H, and alkyl, an aryl or a heteroaryl,
R2 and R3 represent either independently of each other, an H, an alkyl, an aryl or a heteroaryl, or R2 and R3 are bridged within a same ring or via several rings;
R5 represents CF₃, (CF₂)ₙCF₃ or CF₂H with “n” representing an integer comprised between 1 and 7.

2. The compound, according to claim 1, wherein:
R1 represents H, a linear or branched C₁-C₁₀ alkyl, or an aryl selected from among the phenyl group C₆H₅, the tolyl group C₆H₄CH₃, the xylyl group C₆H₃(CH₃)₂, the naphthyl group C₁₀H₇, the 4-methoxyphenyl group C₆H₄OCH₃, the 3,4-dimethoxyphenyl group C₆H₃(OCH₃)₂, and the group 4-(n-heptyloxyphenyl) group C₆H₄O(CH₂)₆CH₃;
R₂ and R₃ represent, either independently of each other, H, a linear or branched C₁-C₁₀ alkyl, and/or functionalized, an aryl selected from the phenyl group C₆H₅, the tolyl group C₆H₄CH₃, the xylyl group C₆H₃(CH₃)₂, the naphthyl group C₁₀H₇, or a heteroaryl selected from among pyridinyl, pyridazinyl, pyrimidyl, triazinyl, pyrrolyl, pyrazolyl, imidazolyl, (1,2,3)- and (1,2,4)-triazolyl, pyrazinyl, pyrimidinyl, tetrazolyl, furyl, thienyl, isoxazolyl, thiazolyl, phenyl, and oxazolyl, or R₂ and R₃ are bridged within a same ring with 5 or 6 carbon atoms;
R_{F} represents CF₃, (CF₂)ₙCF₃ or CF₂H with n representing an integer comprised between 1 and 7.

3. The compound, according to claim 1, being selected from among the list of the following compounds:
N₂-methyl-4-(trifluromethyl)-6-(4′-methoxyphenyl)-4,5-dihydropyridazin-3(2H)-one
4-(trifluromethyl)-6-(3′,4′-dimethoxyphenyl)pyridazin-3(2H)-one
6-(4′-difluoromethoxy)phenyl)-4-(trifluromethyl)-4,5-dihydropyridazin-3(2H)-one
6-(4′-difluoromethoxy)phenyl)-4-(trifluromethyl)pyridazin-3(2H)-one
2-phenyl-6-(p-tolyl)-4(trifluromethyl)-4,5-dihydropyridazin-3(2H)-one
4-(trifluromethyl)-2-phenyl-6-(p-tolyl)pyridazin-3(2H)-one
6-(4′-(difluoromethoxy)-3′-methoxy-phenyl)-4-(trifluromethyl)-4,5-dihydropyridazin-3(2H)-one
6-(4′-(difluoromethoxy)-3′-methoxy-phenyl)-4-(trifluromethyl)pyridazin-3(2H)-one.

4. The compound, according to claim 1, being used as a inhibitory drug of phosphodiesterases of type IV.

5. The compound, according to claim 1, being in the treatment of obstructive chronic obstructive pulmonary diseases.

6. The compound, according to claim 1, being in the treatment of asthma.

7. The compound, according to claim 1, being in the treatment of cystic fibrosis.

8. The compound, according to claim 1, being an active ingredient of a drug against chronic obstructive pulmonary diseases.

9. A method for treating a chronic obstructive pulmonary disease, the method comprising the steps of:
forming a compound according to claim 1; and
applying the compound for therapeutic use for chronic obstructive pulmonary diseases.
